(19) United States
(12) Patent Application Publication
(10) Pub. No.: US 2015/0047086 A1
(43) Pub. Date: Feb. 19, 2015

(54) Body Shades

(71) Applicant: Mervin Lee Mayes, New York, NY (US)

(72) Inventor: Mervin Lee Mayes, New York, NY (US)

(21) Appl. No.: 14/461,504

(22) Filed: Aug. 18, 2014

Related U.S. Application Data
(60) Provisional application No. 61/957,575, filed on Aug. 19, 2013.

(51) Int. Cl. A42B 1/18; A61F 9/04; A42B 1/00

(52) U.S. Cl. CPC A42B 1/18; A42B 1/00; A61F 9/045
USPC 2/10; 2/209.3

(57) ABSTRACT

A comprehensive body-covering garment which includes a headband/sunglasses combination to which a body-covering veil is attached

The present invention relates to a comprehensive body-covering garment which includes a headband/sunglasses combination to which a body-covering veil is attached.

### BACKGROUND OF THE INVENTION

People enjoy being in the sun but compelling scientific evidence establishes that the sun is harmful to skin and may cause many undesirable conditions such as various skin cancers as well as dryness and premature aging. It is thus increasingly recognized that it is important to protect the skin from sun exposure.

While many types of garments are useful in shielding at least part of the body from the sun, they leave the other portions exposed. For example, a shirt-type garment may protect the torso, but the head, neck and legs are still unprotected, requiring additional garments. A hat and pants may be added to the mix, but these can cause the wearer to feel much too warm. A garment with long sleeves may protect the arms but will start to feel increasingly restrictive the longer the wearer is in the sun.

The present invention overcomes these problems and provides a protective and comfortable garment.

### SUMMARY OF THE INVENTION

The present invention is a combination garment which is comprised of a headband, sunglasses, and a body-covering veil. The sunglasses are held in the headband, protecting the wearer's eyes from the sun, and body-shielding material is attached to the headband so that it hangs down over the ears, neck, torso, arms, and legs. Slits are provided to allow the wearer the ability to extend arms through the material, and the garment is fastened in front for easy donning and doffing.

### DESCRIPTION OF THE DRAWINGS

FIG. 1 is a view of the inventive garment in use.

FIG. 2 is a perspective exterior view of the inventive headband on the head of a user without the veil attached.

FIG. 3 is an interior view of the opened headband without the veil attached.

FIG. 4 is a plan view of the veil before attachment to the headband and prior to stitching the back portions together.

### DETAILED DESCRIPTION OF THE INVENTION

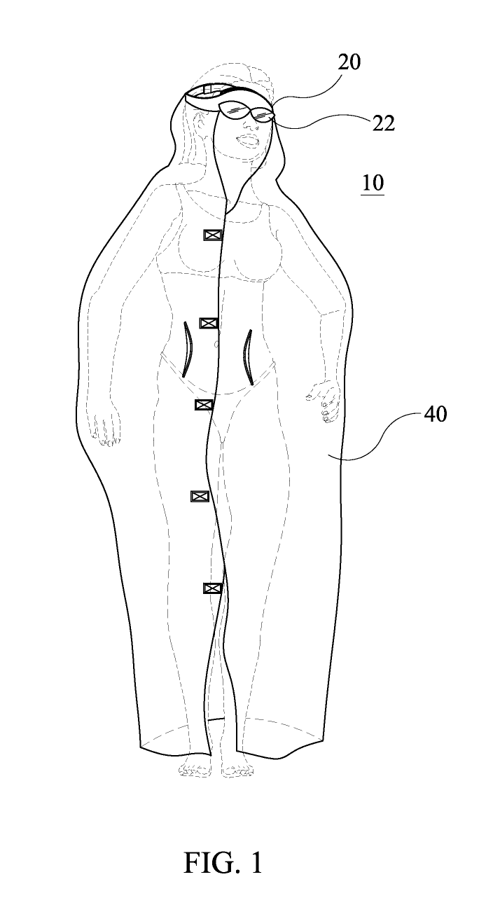

The drawings show the garment of the present invention. FIG. 1 shows a woman wearing a preferred embodiment of the inventive garment 10 where headband 20 is on the user's head, sunglasses 22 are held in headband 20, and veil 40 is attached to headband 20 and covers the wearer's body.

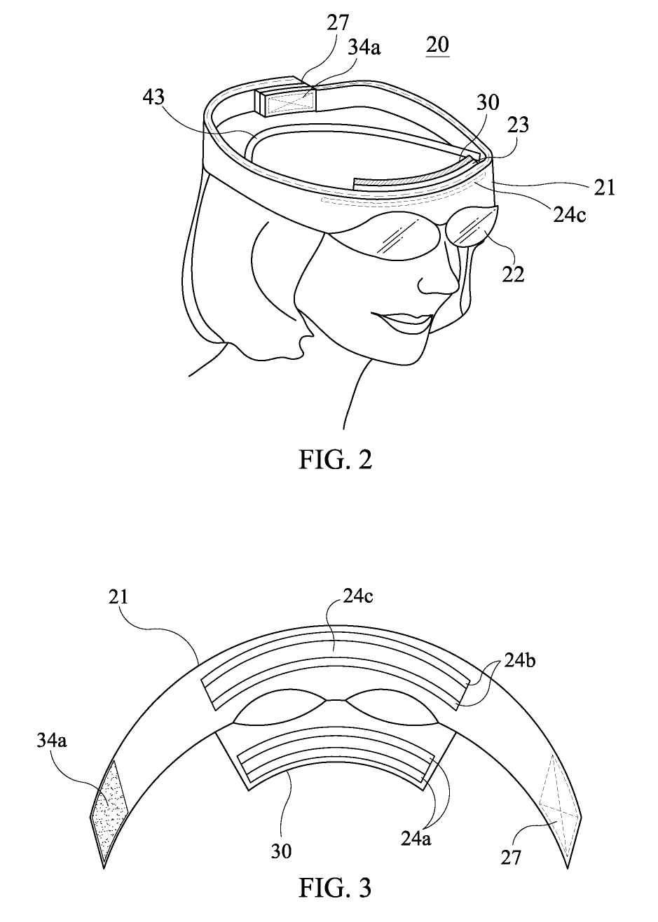

Headband 20 is shown in FIG. 2. Headband 20 encircles the head of the wearer. It is formed from main fabric strip 21 and includes sunglasses 22 held in place in a retainer pocket 23. Fabric strip 21 is preferably an elastic and absorbent material, such as an elasticized terrycloth. Flap 30, shown in FIG. 3, depends from main fabric strip 21 and has at least one attachment strip 24a which, when flap 30 is folded upward toward attachment strip 24b, connects thereto and forms retainer pocket 23. Flap 30 can be made of a microfiber or some other material typically used as clothing liners.

Attachment strips 24a and 24b can be velcro or some other two-part fastening device. Headband 27 can be of an unopened circular configuration or can be a split circle, where the circle is split at the back of the headband and fasteners 27, which can be velcro or some other fastening device, provide circle closure. One side of another fastening device 34a, is attached to the interior portion of strip 21, where it will be at the back of the head of the user.

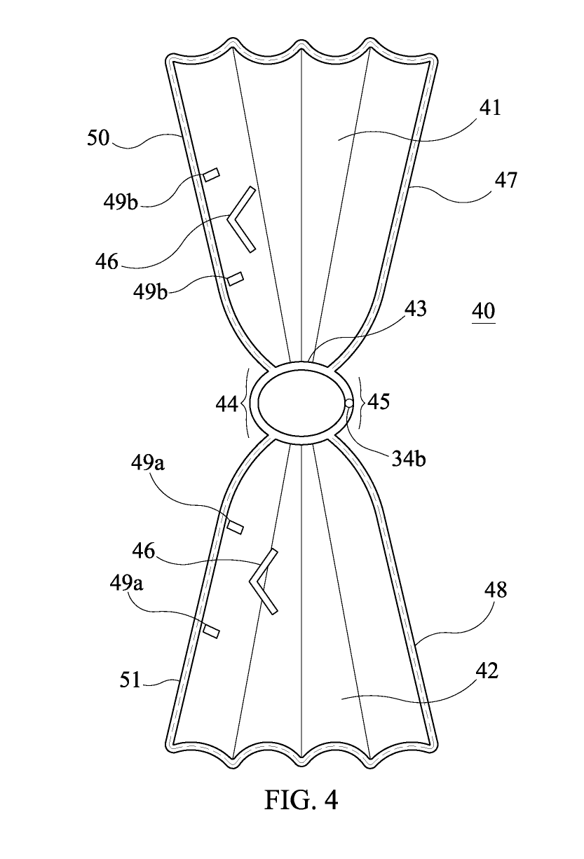

Veil 40 of the garment is constructed from two fabric pieces, 41 and 42, shown in FIG. 4, which are hemmed over opposite sides of an expanding material band 43. The portion 44 of band 43 which is to be above the face is not covered by pieces 41 or 42; likewise, the portion 45 at the back of the head is not covered by pieces 41 or 42. Pieces 41 and 42 each have an arm slit 46. After hemming over band 43, the pieces are stitched together along sides 47 and 48 to close the back of garment 10. Closures 49a and 49b are attached to the front opening of the veil 41 which is formed by sides 50 and 51. Fastener 34b, which can be a velcro portion or some other fastening device, is attached to portion 45.

Portion 44 of band 43 is then placed into pocket 23 and flap 30 is attached to strip 21. Fastener 34b is attached to fastener 34a. In this way, garment 10 is constructed.

Garment 10 is worn by placing headband 20 on the head with sunglasses 22 over the eyes and by closing fastener 27. Veil 40 is allowed to drape over the body.

The material to be used for veil 40 should be lightweight, breathable, and quick-drying as well as capable of providing ample protection from ultraviolet radiation. Commercially available sprays that provide UV protection can be used to treat the fabric. Cooling assistance fabrics are also appropriate for use in the invention.

1. A sun-protective garment comprising:
a headband having a front part and a rear part, wherein a retainer pocket is located in the front part and a first fastening device is attached to the rear part;
a substantially circular elastic material band having a first, a second, a third, and a fourth portion, wherein the first and third portions are diametrically opposed to each other on the material band and the second and fourth portions are diametrically opposed to each other on the material band;
a first piece and second piece of veiling which are attached to the first and third portions, respectively, of the material band and wherein the first piece and second piece of veiling each have a front edge and a rear edge, and the rear edge of the first piece is sewn to the rear edge of the second piece, and the front edges of the pieces of veiling can be attached to each other by a second fastening device;
whereby the second portion of the material band is placed into the retainer pocket and the fourth portion of the material band is attached to the first fastening device.

2. A garment as claimed in claim 1 further comprising a pair of sunglasses fit into the retainer pocket whereby a pair of lenses of the sunglasses protrude downward through the headband.

3. A garment as claimed in claim 1 further comprising a first arm slit through the first piece of veiling and a second arm slit on the second piece of veiling.

4. A sun-protective garment comprising a headband, sunglasses extending from the headband over a wearer's eyes, and a veil that is attached to the headband and covers the wearer's body.
